SEQUENCE LISTING (2 sequences)

SEQ ID NO: 1 - protein, 449 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, Artificial Sequence
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0016753 A1
(43) Pub. Date: Jan. 17, 2019

(54) METHODS FOR SEPARATING ISOFORMS OF MONOCLONAL ANTIBODIES

(71) Applicant: Oncobiologics, Inc., Cranbury, NJ (US)

(72) Inventors: Eun JANG, Cranbury, NJ (US); Pradeep PANDEY, Cranbury, NJ (US); Kaushal JERAJANI, Cranbury, NJ (US); Scott GANGLOFF, Cranbury, NJ (US)

(21) Appl. No.: 16/067,196

(22) PCT Filed: Jan. 6, 2017
(86) PCT No.: PCT/US2017/012477

§ 371 Date: Jun. 29, 2018

Related U.S. Application Data
(60) Provisional application No. 62/276,378, filed on Jan. 8, 2016.

(51) Int. Cl. C07K 1/18; C07K 16/32; C07K 16/06

(52) U.S. Cl. CPC C07K 1/18; C07K 16/32; C07K 2317/41; C07K 2317/14; C07K 16/065

(57) ABSTRACT

Charge variants of a recombinantly expressed antibody population may be separated both from the main antibody molecule and from each other. Separation and isolation of charge variants may proceed via a combined modulation of salt concentration and pH during charge variant elution from a cation exchange support. Isolated charge variants may be assessed for their contribution to the potency of the overall antibody preparation. The make-up of an antibody preparation, at least in terms of the proportion of charge variants and of the main antibody can thus be controlled, for example, for biosimilar matching or for improving potency of the preparation.

### RELATED APPLICATIONS

This application claims priority to U.S. Patent Application No. 62/276,378, filed Jan. 8, 2016, the contents of which are herein incorporated by reference in their entirety.

### FIELD OF THE INVENTION

The invention relates generally to the field of protein biochemistry and analytical chemistry. More particularly, the invention relates to an analytical chromatography process for fractionating charge variants of monoclonal antibodies, which provides for an enriched or more homogenous antibody preparation, and which also provides for the removal of charge variants that diminish the potency of the antibody preparation.

### INCORPORATION-BY-REFERENCE OF SEQUENCE LISTING

The contents of the text file named “ONBI-007001WO_SeqList.txt”, which was created on Jan. 4, 2017 and is 6 KB in size, are hereby incorporated by reference in their entirety.

### BACKGROUND OF THE INVENTION

Various publications, including patents, published applications, accession numbers, technical articles and scholarly articles are cited throughout the specification. Each of these cited publications is incorporated by reference herein, in its entirety and for all purposes.

Monoclonal antibodies (mAbs) may be used as therapeutic proteins. Purified monoclonal antibodies are most often present in a complex heterogeneous mixture. Monoclonal antibodies have charge heterogeneity that optimizes the balance of gaining favorable electrostatic interactions and determines their structure, stability, binding affinity, chemical properties and, hence, their biological activity. There are forms of heterogeneity that occur during protein expression and manufacture caused by enzymatic processes or spontaneous degradation and modifications.

Antibodies undergo chemical modification via several different mechanisms, including oxidation, deamidation, glycation, isomerization and fragmentation that result in the formation of various charge variants and heterogeneity. Chemical and enzymatic modifications such as deamidation, and sialylation, result in an increase in the net negative charge on mAbs and cause a decrease in pI values, thereby leading to formation of acidic variants. C-terminal lysine cleavage results in the loss of net positive charge and leads to formation of the main antibody or acidic variants. Another mechanism for generating acidic variants is the formation of various types of covalent adducts, e.g., glycation, where glucose or lactose can react with the primary amine of a lysine or arginine residue during manufacturing in glucose-rich culture media or during storage if a reducing sugar is present in the formulation. Formation of the basic variants can result from the presence of one or more C-terminal lysines or proline amidation, succinimide formation, amino acid oxidation or removal of sialic acid, which introduce additional positive charges or removal of negative charges; both types of modifications cause an increase in pI values.

Due to the probable impact on potency, post-translational modification (PTM) such as deamidation of asparagine, isomerization of aspartic acid, and methionine-oxidation should be assessed. But PTM and potency analyses of intact molecules generally provide limited information given the complexity of the biologics therapeutics such as mAbs. Accordingly, there remains a need to single out and assess variant molecules for their effect on the preparation on the whole, particularly with respect to a structure-function correlation.

### SUMMARY OF THE INVENTION

The disclosure features methods for separating isoforms of recombinantly expressed antibodies. Separation may allow, for example, assessment of the relationship between the antibody structure and function. Such isoforms include acidic charge variants, basic charge variants, and the main antibody. The isoforms are of monoclonal antibodies.

In general, provided herein are methods for separating isoforms of recombinantly expressed antibodies involving loading a recombinantly expressed antibody preparation including an antibody and a plurality of charge variants of the antibody onto a cation exchange chromatography support containing a ligand capable of capturing the antibody and the charge variants, and fractionating the charge variants by passing a first mobile phase buffer containing from about 20 mM to about 30 mM MES and having a pH of about 6.1 through the support and, while the first mobile phase buffer is being passed through the support, adding a second mobile phase buffer containing from about 40 mM sodium phosphate to about 60 mM sodium phosphate and about 95 mM sodium chloride and having a pH of about 8.0 to the first mobile phase buffer to achieve a mixture of about 90% by volume of the first mobile phase buffer and about 10% by volume of the second mobile phase buffer, gradient eluting one or more of the charge variants from the ligand by gradually increasing the amount of the second mobile phase buffer in the mixture to achieve about 55% by volume of the first mobile phase buffer and about 45% by volume of the second mobile phase buffer, and collecting the one or more charge variants into separate fractions.

In some preferred embodiments, the antibody includes trastuzumab and charge variants and/or isoforms thereof. By way of non-limiting example, trastuzumab can include a heavy chain having the amino acid sequence of SEQ ID NO: 1 and a light chain having the amino acid sequence of SEQ ID NO: 2.

The first mobile phase buffer may contain from about 23 mM to about 25 mM of MES, and, in one preferred embodiment, contains about 24 mM MES. The second mobile phase buffer may contain from about 45 mM to about 55 mM of sodium phosphate, and, in one preferred embodiment contains contain about 50 mM sodium phosphate. In one embodiment, the step of adding the second mobile phase buffer to the first mobile phase buffer to achieve a mixture of about 90% by volume of the first mobile phase buffer and about 10% by volume of the second mobile phase buffer occurs by adding a bolus of the second mobile phase buffer to the first mobile phase buffer to achieve the mixture substantially immediately. In another embodiment, the step of adding the second mobile phase buffer to the first mobile phase buffer to achieve a mixture of about 90% by volume of the first mobile phase buffer and about 10% by volume of the second mobile phase buffer occurs by infusing the second mobile phase buffer into the first mobile phase buffer over a period of time to achieve the mixture.

The isoforms of the antibody elute from the cation exchange (CEX) ligand as the salt concentration and pH increases as the second mobile phase buffer takes on a greater proportion of the CEX flow through. Acidic charge variants, basic charge variants, and the main antibody may be eluted from the column according to this process. The eluted isoforms may be collected in separate fractions. One, two, three, four, five, six, seven, eight, nine, ten, eleven, or more isoforms may be collected into individual fractions, or may be collected in combination into fractions.

In any of the methods disclosed herein, the method can involve collecting two or more of the charge variants into separate fractions; three or more of the charge variants into separate fractions; four or more of the charge variants into separate fractions; five or more of the charge variants into separate fractions; six or more of the charge variants into separate fractions; seven or more of the charge variants into separate fractions; eight or more of the charge variants into separate fractions; nine or more of the charge variants into separate fractions; and/or 10 or more of the charge variants into separate fractions.

According to the methods of the disclosure, the charge variants may include up to six acidic charge variants and up to four basic charge variants.

Each of the separate fractions (i.e., the isoform fraction or the charge variant fraction) is highly purified. For example, one or more of the separate fractions may contain the collected isoform or charge variant (or a combination thereof) at a purity of at least about 90% based on the total weight of the fraction; at least about 91% based on the total weight of the fraction; at least about 92% based on the total weight of the fraction; at least about 93% based on the total weight of the fraction; at least about 94% based on the total weight of the fraction; at least about 95% based on the total weight of the fraction; at least about 96% based on the total weight of the fraction; at least about 97% based on the total weight of the fraction; at least about 98% based on the total weight of the fraction; and/or at least about 99% based on the total weight of the fraction.

By way of non-limiting example, any of the methods of the disclosure may additionally involve eluting the antibody (i.e., trastuzumab) from the ligand.

The purification process may be repeated, following which, fractions may be combined and, optionally, concentrated. For example, another recombinantly expressed antibody preparation containing the antibody and a plurality of charge variants and/or isoforms can be loaded onto the support, the fractionating step can be repeated, and the separate fractions of the same charge variant and/or isoform from each antibody preparation can be pooled together. Likewise, another recombinantly expressed antibody preparation containing the antibody and a plurality of charge variants and/or isoforms can be loaded onto the support, the fractionating step can be repeated, the eluting step can be repeated, and the eluted antibody, charge variants, and/or isoforms thereof can be pooled together.

In one preferred embodiment, the charge variants and/or isoforms being combined are the same charge variants and/or isoforms, though different charge variants and/or isoforms may be combined together, including any of the acidic variants with any other acidic variants, or with any of the basic variants, or with the main antibody, or including any of the basic variants with any other basic variants, or with any of the acidic variants, or with the main antibody. The main antibody may be combined with other fractions of the main antibody, or with any combination of acidic or basic charge variants.

In accordance with any of the methods of the disclosure, one or more of the separate fractions of charge variants and/or isoforms having enhanced potency relative to the antibody itself are pooled together. By way of non-limiting example, the antibody can be pooled together with one or more separate fractions of charge variants and/or isoforms having enhanced potency relative to the antibody.

The combination of isoforms may be used, for example, to modulate or control the potency or therapeutic efficacy of a particular therapeutic antibody formulation. For example, the separated isoform fractions may be combined in order to match the relative percentages of isoforms or isoform categories (e.g., acidic or basic charge variants) of a reference antibody formulation, such as in biosimilar antibody manufacture. The separated isoform fractions may be combined in order to enhance the potency or therapeutic efficacy of an antibody formulation, for example, by excluding isoforms that reduce the potency or therapeutic efficacy of the antibody formulation, such as in biobetter antibody manufacture.

By way of non-limiting example, any of the methods disclosed herein, may additional involve pooling the eluted antibody (e.g., trastuzumab) together with one or more of the separate fractions of charge variants to produce a biosimilar antibody (e.g., trastuzumab) composition having a proportion of antibody (e.g., trastuzumab) and charge variants thereof substantially identical to the proportion of antibody (e.g., trastuzumab) and charge variants thereof in a U.S. Food and Drug Administration-licensed antibody (e.g., trastuzumab) composition.

Also provided are antibody isoforms or combinations thereof, and/or formulations thereof produced according to any methods described or exemplified herein. Suitable formulations may contain the antibody isoforms or combinations thereof along with one or more pharmaceutically acceptable carriers and/or pharmaceutically acceptable excipients. The antibody isoforms or combinations thereof within the formulation have a 90% or greater level of purity, a 91% or greater level of purity, a 92% or greater level of purity, a 93% or greater level of purity, a 94% or greater level of purity, a 95% or greater level of purity, a 96% or greater level of purity, a 97% or greater level of purity, a 98% or greater level of purity, or a 99% or greater level of purity, and/or are substantially free of other antibody isoforms.

Any of the aspects and embodiments described herein can be combined with any other aspect or embodiment as disclosed here in the Summary of the Invention, in the Drawings, and/or in the Detailed Description of the Invention, including the below specific, non-limiting, examples/embodiments of the present invention.

Unless otherwise defined, all technical and scientific terms used herein have the same meaning as commonly understood by one of ordinary skill in the art to which this application belongs. In the specification, the singular forms also include the plural unless the context clearly dictates otherwise.

Although methods and materials similar to or equivalent to those described herein can be used in the practice and testing of the application, suitable methods and materials are described below. All publications, patent applications, patents, and other references mentioned herein are incorporated by reference.

The references cited herein are not admitted to be prior art to the claimed application. In the case of conflict, the present specification, including definitions, will control. In addition, the materials, methods, and examples are illustrative only and not intended to be limiting.

Other features and advantages of the application will become apparent from the following detailed description in conjunction with the examples.

### BRIEF DESCRIPTION OF THE DRAWINGS

FIG. 1 shows the cation exchange chromatography profile of unfractionated trastuzumab.

FIG. 2 shows a purity analysis of cation exchange fractions of trastuzumab charge variants.

FIG. 3 shows an example of a mobile phase gradient, whereby the percentage of the second mobile phase buffer is added to a first mobile phase buffer over time, with the percentage of the second mobile phase buffer indicated.

### DETAILED DESCRIPTION OF THE INVENTION

Various terms relating to aspects of the present invention are used throughout the specification and claims. Such terms are to be given their ordinary meaning in the art, unless otherwise indicated. Other specifically defined terms are to be construed in a manner consistent with the definition provided herein.

As used herein, the singular forms “a,” “an,” and “the” include plural referents unless expressly stated otherwise.

As used herein, the terms “comprising,” “having,” and “including” encompass the more restrictive terms “consisting essentially of” and “consisting of.”

As used herein, “fragments” of monoclonal antibodies include, but are not limited to constant region, variable region, heavy chain, light chain, heavy chain variable region, light chain variable region, heavy chain CDR1, heavy chain CDR2, heavy chain CDR3, light chain CDR1, light chain CDR2, and/or light chain CDR3. “Functionally active” fragments can include any monoclonal antibody fragments that are capable of binding an antigen.

It has been observed in accordance that modulation of the pH of cation exchange (CEX) chromatography elution buffer as coupled to modulation of the salt concentration can be harnessed to elute particular antibody charge variants from the CEX support, in order to separate charge variants from the main antibody in a preparation of recombinantly expressed antibodies. This particular technique may be used to obtain a higher purity of the main (desired) antibody molecule, with the antibody preparation thus including fewer acidic or basic species, or may be used to control the levels of particular variants in the antibody preparation, for example, in order to match a biosimilar antibody preparation to the reference antibody preparation (e.g., in order to pass regulatory scrutiny and maintain status as a biosimilar product). The inclusion of variant species, such as charge species, has implications for ultimate potency of the antibody preparation. Thus, these techniques can be used to determine the positive or negative contributions to overall potency made by particular charge species, such that the preparation may be enriched to include fewer charge species that detract from the potency, and while leaving in place charge species that are neutral or that enhance the overall potency of the antibody preparation. These techniques may be used to modify the processing steps in the purification scheme with an aim, for example, of reducing less potent charge variants in order to manufacture biobetter antibodies, or of keeping the charge variant profiles of biosimilar antibodies as consistent as possible relative to the reference product antibody.

Accordingly, the disclosure features methods for isolating charge variants of a monoclonal antibody recombinantly expressed in a bioreactor. In addition, the disclosure features methods for modulating levels of charge variants in a monoclonal antibody preparation. The processes according to the disclosure are suitable for any recombinantly expressed antibody whose preparation includes charge variants (the terms charge variants and charge species are used interchangeably herein).

In some preferred, non-limiting embodiments, the antibody specifically binds to an epitope on HER-2/neu, and the epitope may be linear or conformational. The charge variant isolation methodology described herein is suitable for full length monoclonal antibodies, which contain both variable and constant regions, and is also suitable for antibody derivatives, as well as fragments and/or portions of a full-length antibody.

In some embodiments, the antibody is trastuzumab. By way of non-limiting example, the antibody may include a heavy chain having the amino acid sequence of SEQ ID NO: 1 and/or a light chain having the amino acid sequence of SEQ ID NO: 2. In one preferred embodiment, the antibody includes a heavy chain constant domain and/or a light chain constant domain.

The antibody is expressed using mammalian cells. Non-limiting examples of suitable mammalian expression hosts include Chinese Hamster Ovary (CHO) cells and human embryonic kidney 293 (HEK293) cells, as well as SP2/0 and NS0 cells. Once expressed, the antibody may be clarified from its mammalian host cells by either a two stage depth filtration or a centrifugation process. Following depth filtration, the material may be passed through a 0.2 μm filter to achieve the clarified cell culture supernatant. The clarified cell culture supernatant, which includes the main (desired) antibody, as well as charge and other variants thereof, along with other cell proteins and soluble cell materials, can then be subject to purification schemes to isolate the main antibody as well as its charge variants.

As a first step, the antibody preparation (at this early point, the clarified cell culture supernatant) may be loaded onto a support containing Protein A, whereby the antibodies interact with the Protein A. The support may contain particles that may be packed into a chromatography column. The Protein A may have an antibody binding capacity of from about 10 g/L to about 100 g/L, from about 10 g/L to about 60 g/L, or from about 20 g/L to about 50 g/L. MabSelect SuRe® Protein A media is an example of a suitable Protein A support. UNOsphere SUPrA™ media, ProSep® Ultra Plus Protein A media, and AbSolute® High Cap Protein A media are other examples of suitable Protein A supports. Any suitable Protein A support available in the art may be used.

Loading of the antibody preparation onto the Protein A support is carried out at a temperature, in a volume, and for a time suitable to allow for maximal adsorption of the monoclonal antibodies to the Protein A ligand. Undesired materials that do not adsorb to the Protein A ligand flow through the support during chromatography, but the antibody and its variants that include an Fc region adhere to the Protein A ligand on the support. To further remove undesired materials that adhere to the ligand or to the antibody protein, the antibody-adsorbed support can be washed. Any suitable number of washes may be used, and the wash may contain a buffer and sufficient stringency to remove undesired materials but not elute antibodies from the Protein A.

Following the wash, the monoclonal antibodies and variants thereof are eluted from the Protein A support. Elution may be carried out at a temperature, in a volume, and for a time suitable to allow for maximal elution yield of the monoclonal antibodies from the Protein A ligand. Elution buffer can be acidic. Elution of the monoclonal antibody produces an eluate containing the monoclonal antibody, as well as variants of the antibody. For the process development or manufacture of trastuzumab, analytical supports such as an accurate determination of the charge profiles as well as the percentage of acidic variants and basic variants in the process control steps are important. An example of the weight percentage breakdown from two separate runs is shown in Table 1.

TABLE 1
Example trastuzumab charge variants results for Protein A eluate.

| Sample Description | % Acidic | % Main | % Basic |
|---|---|---|---|
| Protein Eluate Sample 1 | 40.7 | 44.7 | 14.6 |
| Protein Eluate Sample 2 | 42.7 | 41.9 | 15.4 |

The eluate including the monoclonal antibody optionally may be treated to inactivate any viruses present in the eluate. The virus inactivation may involve acidifying the eluate at a temperature and for a period of time sufficient to inactivate any viruses present in the eluate. The acidification may occur, for example, by adding acetic acid, citric acid, hydrochloric acid, formic acid, or combination thereof to the eluate until a desired pH is achieved. The eluate may be warmed before, during, or after acidification. Once at the desired inactivation temperature, the eluate is maintained at both the pH and temperature for a period of time sufficient to inactivate substantially all latent viruses in the eluate. After this virus inactivation hold time elapses, the pH of the eluate may be increased, for example, by addition of a suitable basic buffer.

Following the virus inactivation step, or following the Protein A elution if virus inactivation is not included, the monoclonal antibody may be further purified with a second chromatography step. During this chromatography step, charge variants may be isolated. The chromatography technique is cation exchange (CEX) chromatography.

The CEX chromatography media may include a support containing a sulfapropyl ligand. A non-limiting example of a suitable media includes Capto® SP ImpRes media. In some embodiments, the chromatography media contains a support containing a carboxymethyl, phosphate, sulfoethyl, or sulfonate ligand. The ligand may be linked to any suitable support, which may include an agarose, ceramic, hydrophilic polymer, polymeric bead, polystyrene-divinyl benzene, or polyvinyl ether support. The support may contain particles that may be packed into a chromatography column.

The eluate from the Protein A chromatography step, which may be the filtered eluate from the virus inactivation step, can be loaded onto the CEX chromatography support and allowed to flow through the support, whereby the antibodies interact with the ligand. Loading of the flow-through pool including the monoclonal antibody onto the CEX support is carried out at a temperature, in a volume, and for a time suitable to allow for maximal adsorption of the monoclonal antibodies to the ligand support. The main antibody molecules, as well as the variants adsorb to the support. Undesired materials that do not adsorb to the ligand support flow through the support during chromatography. To further remove undesired materials that adhere to the ligand, the antibody-adsorbed support may be washed.

The CEX chromatography may be coupled to HPLC in order to better visualize the separation and, ultimately, collect the separated charge variants (and main antibody). Mass loading onto the CEX column may affect the HPLC peak resolution and the yield of the isolated antibody variants. The balance between the yield and the purity of each isolated isoform may be considered in terms of the optimum loading. The protein quantity of about 1 mg per each column loading was observed to provide a decent yield and purity balance.

The acidic charge variants may then be eluted from the CEX ligand, while the main antibody and basic charge variants remain. Following elution of the acidic charge variants, the main antibody is eluted from the CEX ligand, while the basic charge variants remain. Following elution of the main antibody, the basic variants are eluted from the CEX ligand. As each antibody isoform is eluted (successively), it is collected as a separate, purified fraction. The isolated charge variants are collected as fractions that are substantially free of other charge variants as well as the main antibody molecule.

Fractionating charge variants of the antibody can combine CEX and HPLC techniques, and utilizes two mobile phases to change buffer conditions in order that each charge variant may be successively eluted from the CEX ligand. The second mobile phase includes higher salt and a higher pH relative to the first mobile phase, and the second mobile phase buffer solution is added to the first mobile phase buffer solution in order to establish a salt and pH gradient that elutes charge variants in succession. As charge variants elute, they are collected into individual fractions.

In some embodiments, the first mobile phase buffer contains from about 20 mM to about 30 mM of 2-(N-Morpholino)ethanesulfonic acid (MES). The MES buffer may be prepared as an aqueous combination of MES hydrate (free acid) and MES sodium salt to achieve the desired concentration. MES may be substituted with any suitable buffering agent capable of maintaining the desired pH level, which is from about 5.9 to about 6.3 and preferably is about 6.1. A non-limiting example of an alternative buffer is a sodium acetate and acetic acid buffer. The first mobile phase buffer may contain from about 20 mM to about 28 mM of MES, from about 21 mM to about 27 mM of MES, from about 22 mM to about 26 mM of MES, from about 23 mM to about 25 mM of MES, or about 24 mM of MES, and have a pH of from about 5.9 to about 6.3, from about 6 to about 6.2, or about 6.1. In some embodiments, the first mobile phase buffer contains about 24 mM of MES and has a pH of about 6.1.

The second mobile phase buffer may contain from about 40 mM sodium phosphate to about 60 mM sodium phosphate and about 90 mM to about 100 mM of sodium chloride. Sodium phosphate may be substituted with any suitable buffering agent capable of maintaining the desired pH level, which is from about 7.8 to about 8.2 and preferably is about 8. The second mobile phase buffer may contain from about 45 mM sodium phosphate to about 55 mM sodium phosphate, from about 46 mM sodium phosphate to about 54 mM sodium phosphate, from about 47 mM sodium phosphate to about 53 mM sodium phosphate, from about 48 mM sodium phosphate to about 52 mM sodium phosphate, from about 49 mM sodium phosphate to about 50 mM sodium phosphate, or about 50 mM of sodium phosphate, and from about 91 mM to about 99 mM of sodium chloride, 92 mM to about 98 mM of sodium chloride, 93 mM to about 97 mM of sodium chloride, 94 mM to about 96 mM of sodium chloride, or about 95 mM of sodium chloride, and have a pH of from about 7.8 to about 8.2, from about 7.9 to about 8.1, or about 8. In some embodiments, the second mobile phase buffer contains about 50 mM of sodium phosphate and about 95 mM of sodium chloride, and has a pH of about 8. A non-limiting example of a suitable second mobile phase buffer is a Trizma HCl-Trizma base buffer, which may be used in place of sodium phosphate.

Prior to elution of the charge variants, the first mobile phase buffer is passed through the CEX support. As the first mobile phase buffer is passed through the CEX support, the second mobile phase buffer is added to the first mobile phase buffer until the mixture of these buffers is about 90% by volume of the first mobile phase buffer and about 10% by volume of the second mobile phase buffer. The buffers may be mixed together as a bolus, for example, by adding the second mobile phase buffer to substantially immediately reach the 90% to 10% ratio. Alternatively, the buffers may be more gradually mixed together by infusing the second mobile phase buffer into the first mobile phase buffer to go from 100% of the first mobile phase buffer to the mixture of 90% of the first mobile phase buffer and 10% of the second mobile phase buffer over a period of time, usually a few minutes. The 90%-10% mixture is flowed through the support for a time sufficient to equilibrate the support with this buffer combination, usually a few minutes.

To elute the charge variants, the amount (by volume) of the second mobile phase buffer is increased over a gradient, by increasing the amount of the second mobile phase buffer and decreasing the amount of the first mobile phase buffer flowing through the CEX support. As the salt and the pH in the flow-through liquid increase, the charge variants begin to elute from the CEX ligand in succession; first the acid charge variants, followed by the main antibody (non-variant), and then followed by the base charge variants. Each variant, acid or base, and the main antibody, may be identified and collected as a fraction as it elutes.

The charge variants elute in succession via a gradient elution, as the amount of the second mobile phase buffer increases (by volume). The volume of the second mobile phase buffer is increased, over a gradient, from about 10% to about 45%. The last basic variant elutes from the CEX ligand when the second mobile phase buffer is at about 45% by volume and the first mobile phase buffer is at about 55% by volume. The gradient proceeds over a period of time, generally a few minutes. A non-limiting example of suitable gradient time is listed in Table 2 below (times and amounts are approximate).

TABLE 2
Example two step gradient in volume percentage of the first (A) and
second (B) mobile phase buffers.

| Time | Mobile phase A | Mobile phase B |
|---|---|---|
| (approx. min) | (approx. %) | (approx. %) |
| 0 | 90 | 10 |
| 5 | 70 | 30 |
| 30 | 45 | 45 |
| 31 | 5 | 95 |
| 34 | 5 | 95 |
| 35 | 90 | 10 |
| 45 | 90 | 10 |

As the percentage of the second mobile phase buffer increases, the pH of the mixture passing through the CEX support increases, as does the concentration of the salt (NaCl). The antibodies sequentially elute from the CEX ligand as the salt and pH increase; first to elute are the acidic charge variants, then the main antibody elutes, followed by the elution of the basic charge variants. The trastuzumab antibody preparation was observed to include at least ten charge variants, of which six were acidic charge variants and four were basic charge variants. Any subset of these ten variants, in addition to the main antibody, may be fractionated and collected according to the methods described or exemplified herein.

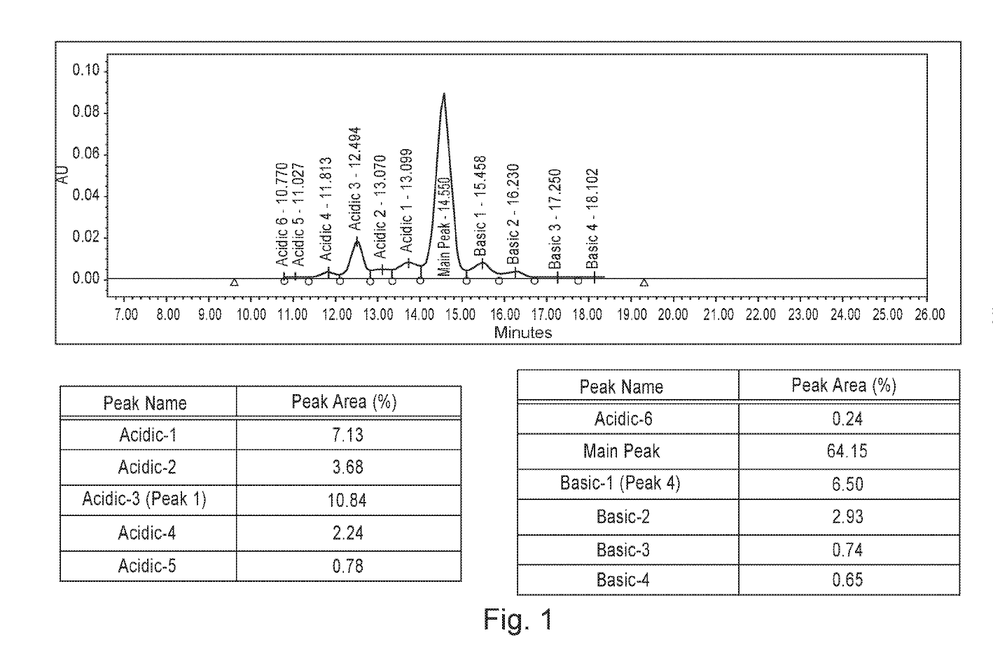

For trastuzumab, the volume percentage of the second mobile phase buffer, as well as the pH and the salt (NaCl) concentrations were determined for the elution of each acidic and basic variant. The CEX profile for the variants and the main antibody is shown in FIG. 1. The elution profile is summarized in Table 3.

TABLE 3
Example mobile phase composition (pH and salt concentration) for
elution of isoforms.

| | Run | | | |
|---|---|---|---|---|
| | Time, | | | NaCl Conc. |
| Peak ID | min. | % MP B | pH | (mM) |
| Acidic-1 (F-5) | 13.5 | 35.1 | 6.8 | 33.4 |
| Acidic-2 (F-4) | 13 | 34.8 | 6.8 | 33.0 |
| Acidic-3 (F-3) | 12.4 | 34.4 | 6.8 | 32.7 |
| Acidic-4 (F-2) | 11.7 | 34.0 | 6.7 | 32.3 |
| Acidic-5 (F-1) | 11 | 33.6 | 6.7 | 31.9 |
| Acidic-6 (F-0) | 10.6 | 33.3 | 6.7 | 31.7 |
| Main Peak (F-6) | 14.5 | 35.7 | 6.8 | 33.9 |
| Basic-1 (F-7) | 15.3 | 36.2 | 6.8 | 34.4 |
| Basic-2 (F-8) | 16 | 36.6 | 6.8 | 34.8 |
| Basic-3 (F-9) | 17.1 | 37.3 | 6.8 | 35.4 |
| Basic 4-4 (F-10) | 17.9 | 37.8 | 6.8 | 35.9 |

In various embodiments, one or more antibody variants; two or more antibody variants; three or more antibody variants; four or more antibody variants; five or more antibody variants; six or more antibody variants; seven or more antibody variants; eight or more antibody variants; nine or more antibody variants; and/or ten or more antibody variants may be fractionated and collected.

It is not necessary that all antibody variants be separated and collected. In some embodiments, only select variants may be separated from the antibody preparation, such as those variants that diminish potency of the overall antibody preparation. Charge variants that enhance potency or are neutral to the potency of the overall antibody preparation may be retained. Potency contributions from charge variants are measured relative to the potency of the main antibody.

Each variant that is collected is substantially pure, and is substantially free of other charge variants as well as the main antibody. The collected charge variant has a purity of at least about 90%. For example, at least about 90% by weight of the material such as the protein content collected in the fraction is the charge variant. The collected charge variant has a purity of at least about 91%, 92%, 93%, 94%, 95%, 96%, 97%, 98%, and/or 99%. Such percentages are based on the weight of material such as protein collected in the fraction.

The fractionation and isolation of charge variants, as well as the main antibody, according to the techniques described and exemplified herein may be repeated any suitable number of times, for example, with multiple cell culture-expressed antibody preparations. Repeating the process collectively increases the overall yield of each antibody isoform, which is desirable, for example, for formulating the antibody as a therapeutic agent. Thus, fractions of antibodies may be combined together. Combined fractions may optionally be concentrated according to any procedure suitable in the art.

In some preferred embodiments, only purified fractions of the main antibody are combined together such that the resultant preparation is substantially depleted of acid and basic charge variants. In some embodiments, only purified fractions of a particular acidic or basic charge variant are combined together—for example, one fraction of purified acid variant 1 is combined with another fraction of purified acid variant 1, but not other acid variants or other basic variants, or the main antibody. By combining fractions of the same charge variant together, the particular charge variant may be tested for potency relative, for example, to the main antibody or to the other charge variants of the antibody. Fractions of select variants may be combined with fractions of other select variants and/or with the fraction of the main antibody. By way of example, but not of limitation, fractions of purified acid variant 1 may be combined with fractions of purified acid variant 4, or fractions of purified acid variant 3 may be combined with fractions of purified acid variant 5 and the main antibody, etc.

Combining variants and/or the main antibody together may be tailored to particular potency values. For example, isolation of the charge variants of an antibody preparation allows the relative affinity, immune effector function, biologic activity, and/or other characteristics of each variant to be assessed individually, such that it may be determined how each charge variant contributes—positively or negatively—to the therapeutic efficacy of the antibody preparation on the whole. Once it is determined whether a charge variant diminishes the potency of the therapeutic antibody preparation, then it may be desirable to isolate such a charge variant during the purification scheme. If it is determined that a particular charge variant enhances the potency of the therapeutic antibody preparation, then it may be desirable to maintain such a charge variant, rather than collect it during the purification scheme or, to the extent that it is isolated and collected, it may be desirable to pool the variant together with the main antibody when preparing the therapeutic antibody formulation. Alternatively, it may be desirable to formulate charge variants with enhanced potency as a separate therapeutic antibody formulation.

In biosimilar manufacture, regulatory agencies such as the U.S. Food and Drug Administration (FDA) or the European Medicines Agency (EMA) may require that the biosimilar antibody preparation maintain the approximate proportion of acidic and/or basic charge variants and main antibody as the reference antibody preparation. Accordingly, the methodologies described and exemplified herein may find utility in matching such proportions, as methodology allows the amount of charge variants in the antibody preparation to be controlled, either through selective elution or by recombining collected fractions. The methodologies may find utility in establishing a biobetter antibody preparation, by selective removal of charge variants that diminish therapeutic efficacy of the antibody preparation and/or by establishing a higher level of purity of the main antibody.

The following examples are provided to describe the invention in greater detail. They are intended to illustrate, not to limit, the invention.

### Example 1

### Materials & Methods

Trastuzumab was expressed recombinantly in a bioreactor cell culture, and initially purified using protein A affinity chromatography. The protein A-purified antibody preparation was then subject to follow-on chromatography purification steps including the cation exchange chromatography. Typically, the materials aliquotted from the in-process control steps are analyzed by analytical CEX chromatography to assess charge variants and separate the desired antibody from these charged isoforms. Dionex was the column manufacturer. The resin for CEX column contains a nonporous core particle with a hydrophilic layer with carboxylated functional group attachment to the core beads.

Separation of the main peak of trastuzumab from acidic and basic charge variants was achieved using an Agilent 1260 Bio-inert HPLC system equipped with a Fraction collector. A semi-prep ProPac™ WCX-10 column with 10 micron particle size and a dimension of 9 mm internal diameter and 250 mm length was used for the isolation of charge variants.

Trastuzumab reconstituted with water for injection (WFI) to approximately 25 mg/mL was used for the isolation of charge isoforms. Mobile phase (MP) A included 24 mM MES buffer at pH 6.1 and mobile phase (MP) B included 50 mM sodium phosphate buffer and 95 mM sodium chloride at pH 8.0. The isoforms were eluted from the column with two step MP gradients from 10 to 30% of phase B in 5 minutes followed by 30 to 45% of phase B in 25 minutes. The column and autosampler temperatures were set at 35° C. and 5° C., respectively. Mobile phase flow was 2.0 mL/min, injection volume was 40 μL, detection was by UV at 280 nm, and the run time was 45 minutes.

An analytical ProPac™ WCX-10 column with 10 micron particle size and a dimension of 4 mm internal diameter and 250 mm length is used for the analysis of charge variants of unfractionated Trastuzumab or the assessment of quantity and purity of isolated charge isoform fractions. The mobile phase compositions and gradients are same as those used for the semi-prep scale except the MP flow that is set at 0.5 mL/min for the analytical column.

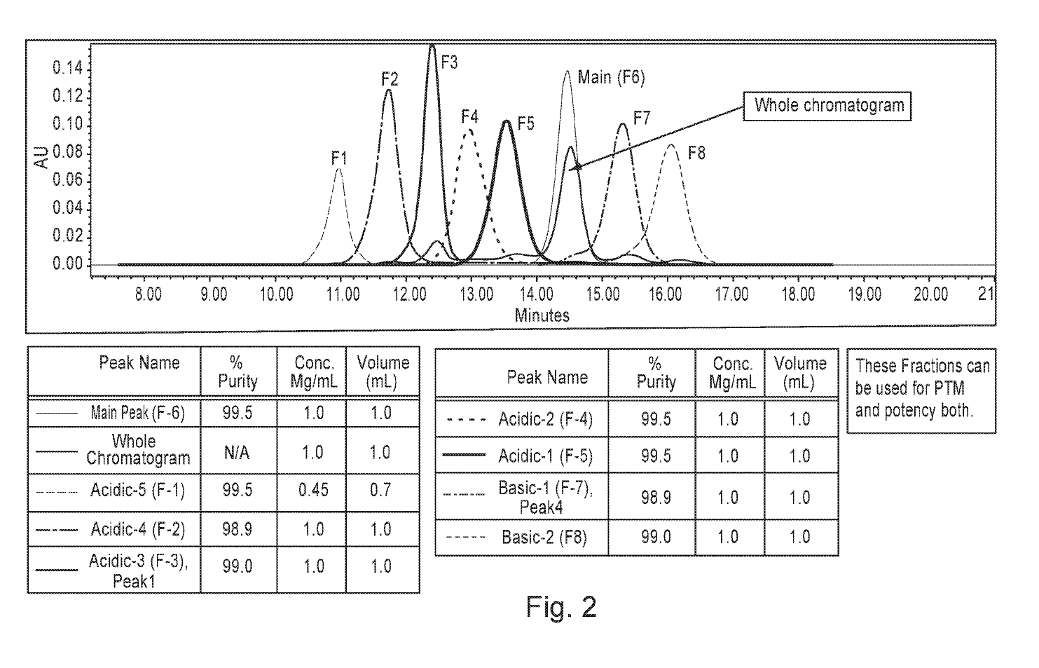

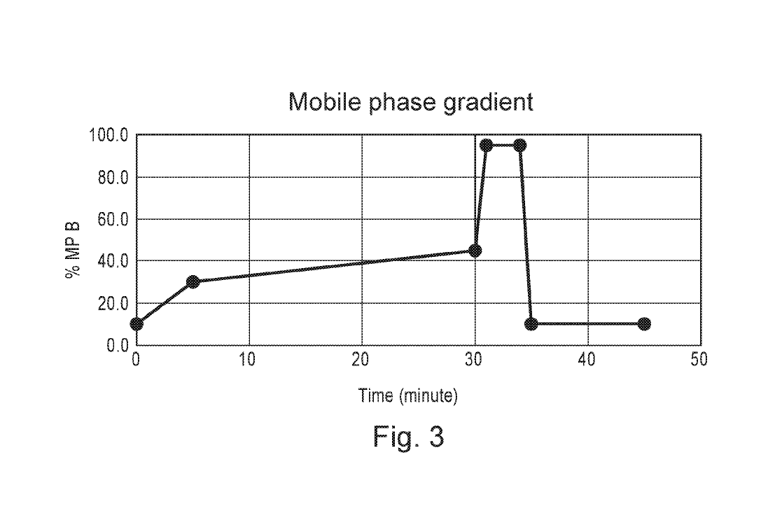

The isolated fractions are buffer exchanged into Trastuzumab formulation buffer for concentration of each isoforms to target concentration of approximately 1 mg/mL. An example CEX chromatography profile for the unfractionated trastuzumab is shown in FIG. 1. An example of an overlaid CEX chromatograms for isolated fractions is shown in FIG. 2. An example of a two-step gradient mobile phase profile for CEX chromatography is shown in FIG. 3.

### EQUIVALENTS

The details of one or more embodiments of the invention are set forth in the accompanying description above. Although any methods and materials similar or equivalent to those described herein can be used in the practice or testing of the present invention, the preferred methods and materials are now described.

The foregoing description has been presented only for the purposes of illustration and is not intended to limit the invention to the precise form disclosed, but by the claims appended hereto.

Sequence Listing

Trastzumab Heavy Chain (SEQ ID NO: 1)
EVQLVESGGG LVQPGGSLRL SCAASGFNIK DTYIHWVRQA
PGKGLEWVAR IYPTNGYTRY ADSVKGRFTI SADTSKNTAY LQMNSLRAED TAVYYCSRWG
GDGFYAMDYW GQGTLVTVSS ASTKGPSVFP LAPSSKSTSG GTAALGCLVK DYFPEPVTVS
WNSGALTSGV HTFPAVLQSS GLYSLSSVVT VPSSSLGTQT YICNVNHKPS NTKVDKKVEP
KSCDKTHTCP PCPAPELLGG PSVFLFPPKP KDTLMISRTP EVTCVVVDVS HEDPEVKFNW
YVDGVEVHNA KTKPREEQYN STYRVVSVLT VLHQDWLNGK EYKCKVSNKA LPAPIEKTIS
KAKGQPREPQ VYTLPPSREE MTKNQVSLTC LVKGFYPSDI AVEWESNGQP ENNYKTTPPV
LDSDGSFFLY SKLTVDKSRW QQGNVFSCSV MHEALHNHYT QKSLSLSPG
Trastzumab Light Chain (SEQ ID NO: 2)
DIQMTQSPSS LSASVGDRVT ITCRASQDVN TAVAWYQQKP
GKAPKLLIYS ASFLYSGVPS RFSGSRSGTD FTLTISSLQP EDFATYYCQQ HYTTPPTFGQ
GTKVEIKRTV AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSFN RGEC

We claim:

1. A method for separating isoforms of recombinantly expressed trastuzumab, comprising
loading a recombinantly expressed trastuzumab preparation comprising trastuzumab and a plurality of charge variants of trastuzumab onto a cation exchange chromatography support comprising a ligand capable of capturing the trastuzumab and the charge variants, and
fractionating the charge variants comprising passing a first mobile phase buffer comprising from about 20 mM to about 30 mM MES and having a pH of about 6.1 through the support, while the first mobile phase buffer is being passed through the support, adding a second mobile phase buffer comprising from about 40 mM sodium phosphate to about 60 mM sodium phosphate and about 95 mM sodium chloride and having a pH of about 8.0 to the first mobile phase buffer to achieve a mixture of about 90% by volume of the first mobile phase buffer and about 10% by volume of the second mobile phase buffer, gradient eluting one or more of the charge variants from the ligand by gradually increasing the amount of the second mobile phase buffer in the mixture to achieve about 55% by volume of the first mobile phase buffer and about 45% by volume of the second mobile phase buffer, and collecting the one or more charge variants into separate fractions.

2. The method according to claim 1, wherein the first mobile phase buffer comprises from about 23 mM to about 25 mM of MES.

3. The method according to claim 1 or claim 2, wherein the first mobile phase buffer comprises about 24 mM of MES.

4. The method according to any one of claims 1 to 3, wherein the second mobile phase buffer comprises from about 45 mM to about 55 mM of sodium phosphate.

5. The method according to any one of claims 1 to 4, wherein the second mobile phase buffer comprises about 50 mM of sodium phosphate.

6. The method according to any one of claims 1 to 5, wherein the step of adding the second mobile phase buffer to the first mobile phase buffer to achieve a mixture of about 90% by volume of the first mobile phase buffer and about 10% by volume of the second mobile phase buffer comprises adding a bolus of the second mobile phase buffer to the first mobile phase buffer to achieve the mixture substantially immediately.

7. The method according to any one of claims 1 to 5, wherein the step of adding the second mobile phase buffer to the first mobile phase buffer to achieve a mixture of about 90% by volume of the first mobile phase buffer and about 10% by volume of the second mobile phase buffer comprises infusing the second mobile phase buffer into the first mobile phase buffer over a period of time to achieve the mixture.

8. The method according to any one of claims 1 to 7, wherein the method comprises collecting two or more of the charge variants into separate fractions.

9. The method according to any one of claims 1 to 8, wherein the method comprises collecting three or more of the charge variants into separate fractions.

10. The method according to any one of claims 1 to 9, wherein the method comprises collecting four or more of the charge variants into separate fractions.

11. The method according to any one of claims 1 to 10, wherein the method comprises collecting five or more of the charge variants into separate fractions.

12. The method according to any one of claims 1 to 11, wherein the method comprises collecting six or more of the charge variants into separate fractions.

13. The method according to any one of claims 1 to 12, wherein the method comprises collecting seven or more of the charge variants into separate fractions.

14. The method according to any one of claims 1 to 13, wherein the method comprises collecting eight or more of the charge variants into separate fractions.

15. The method according to any one of claims 1 to 14, wherein the method comprises collecting nine or more of the charge variants into separate fractions.

16. The method according to any one of claims 1 to 15, wherein the method comprises collecting ten charge variants into separate fractions.

17. The method according to any one of claims 1 to 16, wherein the charge variants comprise up to six acidic charge variants and up to four basic charge variants.

18. The method according to any one of claims 1 to 17, wherein one or more of the separate fractions comprise the collected charge variant at a purity of at least about 90% based on the total protein weight of the fraction.

19. The method according to any one of claims 1 to 18, wherein one or more of the separate fractions comprise the collected charge variant at a purity of at least about 95% based on the total protein weight of the fraction.

20. The method according to any one of claims 1 to 19, wherein one or more of the separate fractions comprise the collected charge variant at a purity of at least about 98% based on the total protein weight of the fraction.

21. The method according to any one of claims 1 to 20, wherein one or more of the separate fractions comprise the collected charge variant at a purity of at least about 99% based on the total protein weight of the fraction.

22. The method according to any one of claims 1 to 21, further comprising eluting the trastuzumab from the ligand.

23. The method according to any one of claims 1 to 22, further comprising loading another recombinantly expressed trastuzumab preparation comprising trastuzumab and a plurality of charge variants of trastuzumab onto the support, repeating the fractionating step, and pooling together the separate fractions of the same charge variant from each trastuzumab preparation.

24. The method according to claim 22, further comprising loading another recombinantly expressed trastuzumab preparation comprising trastuzumab and a plurality of charge variants of trastuzumab onto the support, repeating the fractionating step, repeating the eluting step, and pooling together the eluted trastuzumab.

25. The method according to any one of claims 1 to 24, further comprising pooling together one or more of the separate fractions of charge variants having enhanced potency relative to trastuzumab.

26. The method according to claim 18 or 25, further comprising pooling the eluted trastuzumab together with one or more of the separate fractions of charge variants having enhanced potency relative to trastuzumab.

27. The method according to claim 22 or 24, further comprising pooling the eluted trastuzumab together with one or more of the separate fractions of charge variants to produce a biosimilar trastuzumab composition having a proportion of trastuzumab and charge variants thereof substantially identical to the proportion of trastuzumab and charge variants thereof in a U.S. Food and Drug Administration-licensed trastuzumab composition.

28. A purified isoform of trastuzumab, produced according to the method of any one of claims 1 to 27.

29. The purified isoform of claim 28, further comprising a pharmaceutically acceptable carrier or pharmaceutically acceptable excipient.
